(19) United States
(12) Patent Application Publication
(10) Pub. No.: US 2007/0155672 A1
(43) Pub. Date: Jul. 5, 2007

(54) Sterilized peritoneal dialysis solutions containing heparin

(76) Inventors: Markus Voges, Bruxelles (BE); Dirk Faict, Assenede (BE)

Correspondence Address: BAXTER HEALTHCARE CORPORATION, 1 BAXTER PARKWAY, DF2-2E, DEERFIELD, IL, 60015, US

(21) Appl. No.: 11/326,236

(22) Filed: Jan. 5, 2006

(51) Int. Cl. A61K 31/727; A61K 31/70; B01D 61/00

(52) U.S. Cl. 514/23; 514/56; 210/646

(57) ABSTRACT

Sterilized dialysis solutions containing glycosaminoglycan, for example Heparin, and methods of making and using same are provided. In an embodiment, the present invention provides different peritoneal dialysis solutions containing Heparin that are stable under sterilization conditions. For example, the solutions are designed to provide all-in-one, ready-to-use sterilized solutions for peritoneal dialysis.

### BACKGROUND

The present invention relates generally to medical treatments. More specifically, the present invention relates to sterilized solutions used for dialysis therapy.

Due to disease, insult or other causes, a person's renal system can fail. In renal failure of any cause, there are several physiological derangements. The balance of water, minerals and the excretion of daily metabolic load are no longer possible in renal failure. During renal failure, toxic end products of nitrogen metabolism (e.g., urea, creatinine, uric acid, and others) can accumulate in blood and tissues.

Kidney failure and reduced kidney function have been treated with dialysis. Dialysis removes waste, toxins and excess water from the body that would otherwise have been removed by normal functioning kidneys. Dialysis treatment for replacement of kidney functions is critical to many people because the treatment is life saving. One who has failed kidneys could not continue to live without replacing at least the filtration functions of the kidneys.

Peritoneal dialysis utilizes a sterile dialysis solution or “dialysate”, which is infused into a patient's peritoneal cavity and into contact with the patient's peritoneal membrane. Waste, toxins and excess water pass from the patient's bloodstream through the peritoneal membrane and into the dialysate. The transfer of waste, toxins, and excess water from the bloodstream into the dialysate occurs due to diffusion and osmosis during a dwell period as an osmotic agent in the dialysate creates an osmotic gradient across the membrane. The spent dialysate is later drained from the patient's peritoneal cavity to remove the waste, toxins and excess water from the patient.

There are various types of peritoneal dialysis therapies, including continuous ambulatory peritoneal dialysis (“CAPD”) and automated peritoneal dialysis. CAPD is a manual dialysis treatment, in which the patient connects the catheter to a bag of fresh dialysate and manually infuses fresh dialysate through the catheter or other suitable access device and into the patient's peritoneal cavity. The patient disconnects the catheter from the fresh dialysate bag and allows the dialysate to dwell within the cavity to transfer waste, toxins and excess water from the patient's bloodstream to the dialysate solution. After a dwell period, the patient drains the spent dialysate and then repeats the manual dialysis procedure. Tubing sets with “Y” connectors for the solution and drain bags are available that can reduce the number of connections the patient must make. The tubing sets can include pre-attached bags including, for example, an empty bag and a bag filled with dialysate.

In CAPD, the patient performs several drain, fill, and dwell cycles during the day, for example, about four times per day. Each treatment cycle, which includes a drain, fill and dwell, takes about four hours.

Automated peritoneal dialysis is similar to continuous ambulatory peritoneal dialysis in that the dialysis treatment includes a drain, fill, and dwell cycle. However, a dialysis machine automatically performs three or more cycles of peritoneal dialysis treatment, typically overnight while the patient sleeps.

With automated peritoneal dialysis, an automated dialysis machine fluidly connects to an implanted catheter. The automated dialysis machine also fluidly connects to a source or bag of fresh dialysate and to a fluid drain. The dialysis machine pumps spent dialysate from the peritoneal cavity, through the catheter, to the drain. The dialysis machine then pumps fresh dialysate from the dialysate source, through the catheter, and into the patient's peritoneal cavity. The automated machine allows the dialysate to dwell within the cavity so that the transfer of waste, toxins and excess water from the patient's bloodstream to the dialysate solution can take place. A computer controls the automated dialysis machine so that the dialysis treatment occurs automatically when the patient is connected to the dialysis machine, for example, when the patient sleeps. That is, the dialysis system automatically and sequentially pumps fluid into the peritoneal cavity, allows for dwell, pumps fluid out of the peritoneal cavity, and repeats the procedure.

Several drain, fill, and dwell cycles will occur during the treatment. Also, a final volume “last fill” is typically used at the end of the automated dialysis treatment, which remains in the peritoneal cavity of the patient when the patient disconnects from the dialysis machine for the day. Automated peritoneal dialysis frees the patient from having to manually perform the drain, dwell, and fill steps during the day.

A number of studies evaluated different therapeutic approaches of Heparins for their intrinsic anti-inflammatory properties. Heparin and glycosaminoglycans modulate the response of inflammatory cells to aggression stimuli, neutralizing super oxide radicals in activated leukocytes, inhibiting released proteins by eosinophile cells, inhibiting leukocyte adhesion to the endothelial cells wall, and preventing peritoneal adherences in some animal models.

Heparin and new low molecular weight Heparins (LMWHs) have been shown to stimulate fibrinolysis in mesothelial cells by selective induction of tissue-plasminogen activator (tPA) but not plasminogen activator inhibitor-1 (PAI-1) synthesis. This means that the mesothelial capacity for fibrinolysis is preserved. Moreover, these drugs can interfere with the process of neoangiogenesis, together with angiogenic growing factors such as bFGF (basic fibroblast growth factor), VEGF (vascular endothelial growing factor) and TF (tissue factor).

In another study, intraperitoneal Heparin diminished advanced glycation end products (AGE) in serum and increased the dwell concentration.

In clinical practice, use of intraperitoneal Heparin to prevent adherences and fibrin deposition in the peritoneal dialysis catheter are well known without modifying systemic coagulation. The safety of intraperitoneal Heparin is well established.

One problem that arises with using Heparin in dialysis treatments, for example, is that Heparin must be added aseptically to the sterile dialysis solutions. This requires extensive training and has an inherent risk of an increased peritonitis incident. Although the use of Heparin or other glycosaminoglycans is known, there is currently no ready-to-use, sterilized solutions containing these compounds.

### SUMMARY

The present invention relates generally to dialysis solutions and methods of making and using same. More specifically, the present invention relates to ready-to-use, sterilized peritoneal dialysis solutions that contain glycosaminoglycans, preferably Heparin.

In an embodiment, the present invention provides a dialysis solution comprising one or more dialysis components and glycosaminoglycan that are combined to form the dialysis solution, wherein the dialysis solution is sterilized after the dialysis component and glycosaminoglycan are combined. By way of example, the sterilization can be performed by a technique such as autoclave, steam and combinations thereof.

In an alternative embodiment, the present invention provides a ready-to-use sterilized dialysis solution comprising one or more dialysis components and Heparin that are combined to form the dialysis solution, wherein the dialysis solution is sterilized after the dialysis component and the Heparin are combined. The dialysis solution can also comprise two or more dialysis components which can be stored and sterilized separately wherein the Heparin is added to at least one of the dialysis components and sterilized with said dialysis component. By way of example, the Heparin can comprise a concentration in the dialysis solution from about 1000 IU/L to about 5000 IU/L, preferably around 2500 IU/L. In an embodiment, the Heparin can be non-fractionated Heparin, low molecular weight Heparin, recombinant low molecular weight Heparin and combinations thereof.

With respect to the dialysis components, by way of example, the dialysis component can comprise one or more osmotic agents, buffers, electrolytes and combinations thereof. The osmotic agents can be, for example, glucose, glucose polymers, modified starch, hydroxyethyl starch, polyols, amino acids, peptides, glycerol and combinations thereof. The buffers can be, for example, bicarbonate, lactic acid/lactate, pyruvic acid/pyruvate, acetic acid/acetate, citric acid/citrate, an intermediate of the KREBS cycle and combinations thereof.

In an embodiment, the sterilized dialysis solution can have a pH ranging from 4.5-8. In part, the pH can be adjusted by use of an acid such as lactic acid/lactate, pyruvic acid/pyruvate, acetic acid/acetate, citric acid/citrate, an intermediate of the KREBS cycle, hydrochloric acid and combinations thereof.

In another embodiment, the present invention provides a method of manufacturing a sterilized dialysis solution. For example, the method comprises providing a dialysis component and providing Heparin. The Heparin can be mixed with the dialysis component and the mixture of the dialysis component and the Heparin sterilized to form the sterilized dialysis solution.

In a still further embodiment, the present invention provides a method of manufacturing a sterilized solution. For example, the method comprises providing two or more dialysis components. The dialysis components can include osmotic agents, buffers and electrolytes and combinations thereof. Heparin can be added to one or more of the dialysis components and sterilized with that dialysis component after the Heparin has been added.

In an embodiment, the method comprises separately storing the sterilized dialysis components. The sterilized dialysis components can be combined to form a ready-to-use dialysis solution prior to or during dialysis treatments. By way of example, the dialysis component can be an osmotic agent such as glucose, glucose polymers, modified starch, hydroxyethyl starch, polyols, amino acids, peptides, glycerol and combinations thereof. The dialysis component can also comprise an acid such as lactic acid/lactate, pyruvic acid/pyruvate, acetic acid/acetate, citric acid/citrate, hydrochloric acid, an intermediate of the KREBS cycle and combinations thereof.

In yet another embodiment, the present invention provides a method of providing dialysis to a patient. For example, the method comprises providing an osmotic agent, a buffer and an electrolyte; mixing Heparin with at least one of the osmotic agent, the buffer and the electrolyte to form a dialysis mixture; sterilizing the dialysis mixture; and providing a dialysis solution including the dialysis mixture to the patient. Alternatively, the Heparin can be mixed with all of the osmotic agent, buffer and electrolyte before sterilization.

An advantage of the present invention is to provide improved dialysis solutions.

Another advantage of the present invention is to provide ready-to-use sterilized dialysis solutions containing Heparin.

Yet another advantage of the present invention is to provide improved dialysis solutions and methods using same containing Heparin.

A still further advantage of the present invention is to provide improved methods of making and using sterilized dialysis solutions containing Heparin.

Additional features and advantages are described herein, and will be apparent from, the following Detailed Description.

### DETAILED DESCRIPTION

The present invention generally relates to dialysis solutions. More specifically, the present invention relates to sterilized peritoneal dialysis solutions and methods of making and using same. For example, the dialysis solutions of embodiments of the present invention are designed to provide all-in-one, ready-to-use sterilized dialysis solutions containing at least one glycosaminoglycan, preferably Heparin.

In an embodiment, the present invention provides different peritoneal dialysis solutions containing glycosaminoglycans that are stable under sterilization conditions. The sterilization techniques can be, for example, autoclaving and steam sterilization. For example, the dialysis solutions are sterilized after one or more dialysis components and glycosaminoglycan are combined. As used herein, glycosaminoglycans include Heparin, chondroitin sulfate, sulodioxide dermatan sulfate, hyaluronic acid, Heparan sulfate, and keratan sulfate. The Heparin can be, for example, non-fractionated Heparin, low molecular weight Heparin, and recombinant low molecular weight Heparin and combinations thereof. In the present specification, the term “recombinant” should be understood to mean being produced in a genetically modified manner. For example, the recombinant low molecular weight Heparin can be produced in genetically modified bacteria by conventional techniques of genetic engineering.

In an alternative embodiment, the present invention provides different sterilized dialysis solutions comprising one or more dialysis components and glycosaminoglycan that are combined to form the dialysis solution. For example, the dialysis solution is sterilized after the dialysis component and glycosaminoglycan are combined. By way of example, the glycosaminoglycan, for example Heparin, can comprise a concentration in the dialysis solution from about 1000 IU/L to about 5000 IU/L, preferably around 2500 IU/L.

In addition to the glycosaminoglycan, the sterilized dialysis solutions of the present invention can include any suitable number, type and amount of dialysis components that are typically used as part of, or during, dialysis treatments. By way of example, the dialysis components can comprise one or more suitable osmotic agents, buffers, electrolytes and combinations thereof. Examples of osmotic agents include glucose, glucose polymers, modified starch, hydroxyethyl starch, polyols, amino acids, peptides, glycerol and/or the like and combinations thereof. Examples of the buffers include bicarbonate, lactic acid/lactate, pyruvic acid/pyruvate, acetic acid/acetate, citric acid/citrate, an intermediate of the KREBS cycle and/or the like and combinations thereof. Examples of electrolytes include calcium, magnesium, sodium, potassium, chloride and/or the like and combinations thereof.

In an embodiment, the sterilized dialysis solution has two or more dialysis components. These two or more components can be separately sterilized and stored. For example, the Heparin can be added to at least one of the dialysis components and sterilized with that dialysis component. The dialysis components not containing Heparin can also be sterilized. The dialysis components can be stored separately, for example, in separate compartments or chambers, and combined prior to or during dialysis treatment. Alternatively, the sterilized dialysis components can be combined to form a ready-to-use dialysis solution.

The peritoneal dialysis solutions preferably contain a dialysis component such as an osmotic agent to maintain the osmotic pressure of the solution higher than the physiological osmotic pressure (e.g. higher than about 285 mOsmol/kg). For example, glucose is a preferred osmotic agent because it provides rapid ultrafiltration rates. Other suitable types of osmotic agents can be used in addition to or as a substitute for glucose. The dialysis solution can be subsequently sterilized after the osmotic agent and the Heparin are combined.

Another family of compounds capable of serving as osmotic agents in peritoneal dialysis solutions is that of glucose polymers or their derivatives, such as icodextrin, maltodextrins, hydroxyethyl starch, and the like. While these compounds are suitable for use as osmotic agents, they can be sensitive to low and high pH, especially during sterilization and long-term storage. Glucose polymers, such as icodextrin, can be used in addition to or in place of glucose in peritoneal dialysis solutions. In general, icodextrin is a polymer of glucose derived from the hydrolysis of corn starch. It has a molecular weight of 12-20,000 Daltons. The majority of glucose molecules in icodextrin are linearly linked with α (1-4) glucosidic bonds (>90%) while a small fraction (<10%) is linked by α (1-6) bonds.

The sterilized dialysis solutions of the present invention can be used in a variety of suitable applications. Preferably, the dialysis solutions are used during peritoneal dialysis, such as during continuous ambulatory peritoneal dialysis, automated peritoneal dialysis, and the like. However, it should be appreciated that the present invention can be used in a variety of different and suitable dialysis therapies to treat kidney failure. Dialysis therapy as the term or like terms are used throughout the text is meant to include and encompass any and all suitable forms of therapies that utilize the patient's blood to remove waste, toxins and excess water from the patient. Such therapies, such as hemodialysis, hemofiltration and hemodiafiltration, include both intermittent therapies and continuous therapies used for continuous renal replacement therapy (CRRT). The continuous therapies include, for example, slow continuous ultrafiltration (SCUF), continuous venovenous hemofiltration (CVVH), continuous venovenous hemodialysis (CVVHD), continuous venovenous hemodiafiltration (CVVHDF), continuous arteriovenous hemofiltration (CAVH), continuous arteriovenous hemodialysis (CAVHD), continuous arteriovenous hemodiafiltration (CAVHDF), continuous ultrafiltration periodic intermittent hemodialysis or the like.

Preferably, the dialysis solutions are used during peritoneal dialysis, such as automated peritoneal dialysis, continuous ambulatory peritoneal dialysis, continuous flow peritoneal dialysis and the like. Further, although the present invention, in an embodiment, can be utilized in methods providing a dialysis therapy for patients having chronic kidney failure or disease, it should be appreciated that the present invention can be used for acute dialysis needs, for example, in an emergency room setting. Lastly, as one of skill in the art appreciates, the intermittent forms of therapy (i.e., hemofiltration, hemodialysis, peritoneal dialysis and hemodiafiltration) may be used in the in center, self/limited care as well as the home settings.

The dialysis components can also include bicarbonates and acids. The bicarbonates can comprise an alkaline solution such that the bicarbonate can remain stable without the use a gas barrier overpouch or the like. The bicarbonate solution can have a pH that ranges from about 8.6 to about 10.0, preferably about 9.0. The pH of the bicarbonate solution part can be adjusted with any suitable type of ingredient, such as sodium hydroxide and/or the like. Illustrative examples of the bicarbonate solution of the present invention can be found in U.S. Pat. No. 6,309,673, entitled BICARBONATE-BASED SOLUTION IN TWO PARTS FOR PERITONEAL DIALYSIS OR SUBSTITUTION IN CONTINUOUS RENAL REPLACEMENT THERAPY, issued on Oct. 30, 2001, the disclosure of which is herein incorporated by reference.

The acids can comprise one or more physiological acceptable acids, such as lactic acid, pyruvic acid, acetic acid, citric acid, hydrochloric acid and the like. The acids can be in a solution having a pH that ranges from about 5.0 or less, about 4.0 or less, about 3.0 or less, about 2.0 or less, about 1.0 or less, and any other suitable acidic pH. The use of an organic acid, such as lactic acid, alone or in combination with another suitable acid, such as a suitable inorganic acid including hydrochloric acid, another suitable organic acid (e.g. lactic acid/lactate, pyruvic acid/pyruvate, acetic acid/acetate, citric acid/citrate) and the like in the acid solution can make the solution more physiologically tolerable according to an embodiment.

It should be appreciated that the dialysis solutions of the present invention can include any other suitable solution ingredients for dialysis treatment in addition to those components described above. The pH of the (mixed) dialysis solutions can have a broad range, preferably 4.5-8.0.

By way of example and not limitation, the following are examples of embodiments of dialysis solutions of the present invention containing glycosaminoglycan that can be sterilized.

### EXAMPLE A

| | Concentrations in the | |
|---|---|---|
| Component | mixed solution | |
| Glucose | 0-50% | |
| | Preferably | |
| | 0-5% | |
| Glucose polymer | 0-10% | |
| Amino Acids | 0-30% | |
| | Preferably | |
| | 0-3% | |
| Peptides | 0-30% | |
| | Preferably | |
| | 0-10% | |
| Calcium | 0.5-2 | mmol/L |
| Magnesium | 0-1 | mmol/L |
| Chloride | 70-110 | mmol/L |
| Sodium | 120-140 | mmol/L |
| Lactate | 0-45 | mmol/L |
| Bicarbonate | 0-40 | mmol/L |
| Potassium | 0-4 | mmol/L |
| Pyruvate | 0-40 | mmol/L |
| Citrate | 0-40 | mmol/L |
| Acetate | 0-40 | mmol/L |
| Heparin | 1000-5000 | IU/L |
| | Preferably | |
| | 2500 | IU/L |
| pH | Preferably | |
| | 4.5-8.0 | |

### EXAMPLE B

| | Concentrations in the | |
|---|---|---|
| Component | mixed solution | |
| Glucose | 0-50% | |
| | Preferably | |
| | 0-5% | |
| Glucose polymer | 0-10% | |
| Amino Acids | 0-30% | |
| | Preferably | |
| | 0-3% | |
| Peptides | 0-30% | |
| | Preferably | |
| | 0-10% | |
| Calcium | 0.5-2 | mmol/L |
| Magnesium | 0-1 | mmol/L |
| Chloride | 70-110 | mmol/L |
| Sodium | 120-140 | mmol/L |
| Lactate | 0-45 | mmol/L |
| Bicarbonate | 0-40 | mmol/L |
| Potassium | 0-4 | mmol/L |
| Pyruvate | 0-40 | mmol/L |
| Citrate | 0-40 | mmol/L |
| Acetate | 0-40 | mmol/L |
| Glycosaminoglycan | 1000-5000 | IU/L |
| | Preferably | |
| | 2500 | IU/L |
| pH | Preferably | |
| | 4.5-8.0 | |

### EXAMPLE C

| | Concentrations in the | |
|---|---|---|
| Component | mixed solution | |
| Glucose | 0-5% | |
| Glucose polymer | 0-10% | |
| Amino Acids | 0-3% | |
| Peptides | 0-10% | |
| Calcium | 1-2 | mmol/L |
| Magnesium | 0-0.75 | mmol/L |
| Chloride | 90-110 | mmol/L |
| Sodium | 130-135 | mmol/L |
| Lactate | 0-45 | mmol/L |
| Bicarbonate | 0-40 | mmol/L |
| Heparin (non fractionated LMWH, | 2000-5000 | IU/L |
| recombinant) | | |
| pH | 4.5-8.0 | |

### EXAMPLE D

| | Concentrations in the | |
|---|---|---|
| Component | mixed solution | |
| Glucose | 1.36-3.86% | |
| Calcium | 1.25-1.75 | mmol/L |
| Magnesium | 0.25-0.75 | mmoL/L |
| Chloride | 95-105 | mmol/L |
| Sodium | 132 | mmol/L |
| Lactate | 10-40 | mmol/L |
| Bicarbonate | 0-30 | mmol/L |
| Heparin (non fractionated LMWH, | 2500 | IU/L |
| recombinant) | | |
| pH | 4.5-8.0 | |

### EXAMPLE E

| | Concentrations in the | |
|---|---|---|
| Component | mixed solution | |
| Icodextrin | 7.5% | |
| Calcium | 1.25-1.75 | mmol/L |
| Magnesium | 0.25 | mmol/L |
| Chloride | 95-105 | mmol/L |
| Sodium | 133 | mmol/L |
| Lactate | 10-40 | mmol/L |
| Bicarbonate | 0-30 | mmol/L |
| Heparin (non fractionated LMWH, | 2500 | IU/L |
| recombinant) | | |
| pH | 4.5-8.0 | |

### EXAMPLE F

| | Concentrations in the | |
|---|---|---|
| Component | mixed solution | |
| Amino acids | 1.1% | |
| Calcium | 1.25-1.75 | mmol/L |
| Magnesium | 0.25 | mmol/L |
| Chloride | 95-106 | mmol/L |
| Sodium | 132 | mmol/L |
| Lactate | 10-40 | mmol/L |
| Bicarbonate | 0-30 | mmol/L |
| Heparin (non fractionated LMWH, | 2500 | IU/L |
| recombinant) | | |
| pH | 4.5-8.0 | |

It should be appreciated that the dialysis solutions of the present invention can be housed or contained in any suitable manner such that the dialysis solutions can be effectively prepared, sterilized, stored and used. It should be appreciated that dialysis solutions of the present invention can be modified in any suitable manner. As discussed previously, various osmotic agents or additives can be added to the peritoneal solutions.

### Experimental Sterlization Examples

By way of example and not limitation, the following examples are illustrative of various embodiments of the present invention and further illustrate experimental testing conducted with dialysis solutions where Heparin was added before or after the sterilization process in accordance with embodiments of the present invention. The sterilization methods used were steam sterilization and autoclaving. However, it should be appreciated that any other suitable sterilization techniques may be used.

In part, the purpose of this study was the evaluation of the stability of Heparin in ready to use peritoneal dialysis (PD) solutions during the regular sterilization process for production. The comparison of the results of the sterilized bags wherein Heparin was added before or after the sterilization process indicate the influence of the sterilization process itself on the stability of Heparin under those conditions. As accepted definition, a change of maximal 10% of the activity is tolerable (see, Trissel, LA., Handbook on Injectable Drugs. 11^{th} ed. Bethesda Md.: American Society of Health-System Pharmacists, 2000) and serves as stability criterion.

### Study Design

The comparison study was performed on DIANEAL®, EXTRANEAL® and PHYSIONEAL® solutions available from Baxter Healthcare Corporation. Heparin (non-fractionated and low molecular weight respectively) was added into the solutions as described in table 1. In case of PHYSIONEAL®, the addition were added either to the buffer or to the electrolyte compartment. The activity of non-fractionated and low molecular weight Heparin were evaluated by blood clotting tests. For evaluating the influence of the sterilization process itself on the stability of Heparin, bags containing Heparin in the solution and then sterilized were compared to bags where the Heparin was added after the sterilization process. Every sample was measured twofold in triplicate.

TABLE 1
Overview of prepared bags

| Solution | Concentrations | |
|---|---|---|
| DIANEAL ® with low molecular weight | 1000/2000/5000 | IU/L |
| Heparin | | |
| EXTRANEAL ® with non-fractionated | 2500 | IU/L |
| Heparin | | |
| EXTRANEAL ® with low molecular | 2500 | IU/L |
| weight Heparin | | |
| PHYSIONEAL ® with non-fractionated | 2500 | IU/L |
| Heparin; | | |
| Buffer compartment | | |
| PHYSIONEAL ® with non-fractionated | 2500 | IU/L |
| Heparin; Electrolyte compartment | | |

### Materials Used

DIANEAL® PD4 (Baxter), 1.36% glucose, 2500 ml.

PHYSIONEAL® 40 (Baxter), 2.27% glucose, 2000 ml.

EXTRANEAL® (Baxter), 2000 ml.

CALPARINE® (Sanofi), syringe (0.2 ml) containing 5000 IU Heparin non-fractionated.

FRAGMIN® (Pharmacia), syringe (0.2 ml) containing 2500 IU low molecular weight Heparin.

### Preparation of Samples

### EXAMPLE1

### DIANEAL® with Low Molecular Weight Heparin

To determine the stability of low molecular weight Heparin in a commercially available standard lactate buffered peritoneal dialysis solution (DIANEAL® PD4, 2500 ml), DIANEAL® was prepared and low molecular weight Heparin was added to result in a concentration of 2000 IU/L per bag. Immediately after addition of the low molecular weight Heparin, solutions with Heparin and solutions without Heparin were steam sterilized under standard conditions. For comparison, the same amount of low molecular weight Heparin was added through the medication port to the sterilized solution not yet containing Heparin after the sterilization process.

To determine stability of lower and higher concentrations of low molecular weight Heparin, bags with a concentration of 1000 IU/L and 5000 IU/L, respectively, were produced in the same manner.

TABLE 2
Composition of Experimental Solution Example 1

| Component | Concentration | |
|---|---|---|
| Sodium | 132 | mmol/L |
| Calcium | 1.25 | mmol/L |
| Magnesium | 0.25 | mmol/L |
| Chloride | 95 | mmol/L |
| Lactate | 40 | mmol/L |
| Glucose | 1.36% | |
| Heparin | 1000, 2000, 5000 | IU/L |
| pH | 5.5 | |

### EXAMPLE 2

### EXTRANEAL® with Non-Fractionated Heparin

To determine the stability of non-fractionated Heparin in a commercially available standard icodextrin containing peritoneal dialysis solution (EXTRANEALS, 2000 ml), EXTRANEALS was prepared and non-fractionated Heparin was added to result in a concentration of 2500 IIU /L per bag. Immediately after addition of the non-fractionated Heparin, solutions with Heparin and solutions without Heparin were steam sterilized under standard conditions. For comparison, the same amount of non-fractionated Heparin was added through the medication port to the sterilized solution not yet containing Heparin after the sterilization process.

TABLE 3
Composition of Experimental Solution Example 2

| Component | Concentration | |
|---|---|---|
| Sodium | 133 | mmol/L |
| Calcium | 1.75 | mmol/L |
| Magnesium | 0.25 | mmol/L |
| Chloride | 96 | mmol/L |
| Lactate | 40 | mmol/L |
| Icodextrin | 7.5% | |
| Heparin | 2500 | IU/L |
| pH | 5.5 | |

### EXAMPLE 3

### EXTRANEAL® with Low Molecular Weight Heparin

To determine the stability of low molecular weight Heparin in a commercially available standard icodextrin containing peritoneal dialysis solution (EXTRANEAL(g, 2000 ml), EXTRANEALS was prepared and low molecular weight Heparin was added to result in a concentration of 2500 IU /L per bag. Immediately after addition of the low molecular weight Heparin, solutions with Heparin and solutions without Heparin were steam sterilized under standard conditions. For comparison, the same amount of low molecular weight Heparin was added through the medication port to the sterilized solution not yet containing Heparin after the sterilization process.

TABLE 4
Composition of Experimental Solution Example 3

| Component | Concentration | |
|---|---|---|
| Sodium | 133 | mmol/L |
| Calcium | 1.75 | mmol/L |
| Magnesium | 0.25 | mmol/L |
| Chloride | 96 | mmol/L |
| Lactate | 40 | mmol/L |
| Icodextrin | 7.5% | |
| Heparin | 2500 | IU/L |
| pH | 5.5 | |

### EXAMPLE 4

### PHYSIONEAL® with Non-Fractionated Heparin Added to Buffer

To determine the stability of non-fractionated Heparin in the buffer compartment of a commercially available bicarbonate/lactate buffered peritoneal dialysis solution (PHYSIONEAL® 40, 2000 ml), PHYSIONEAL® was prepared and non-fractionated Heparin was added to result in a concentration of 2500 IU /L per bag. Immediately after addition of the non-fractionated Heparin, solutions with Heparin and solutions without Heparin were steam sterilized under standard conditions. For comparison, the same amount of non-fractionated Heparin was added through the medication port to the sterilized solution not yet containing Heparin after the sterilization process.

TABLE 5
Composition of Experimental Solution Example 4

| Component | Concentration | |
|---|---|---|
| Sodium | 132 | mmol/L |
| Calcium | 1.25 | mmol/L |
| Magnesium | 0.25 | mmol/L |
| Chloride | 95 | mmol/L |
| Lactate | 15 | mmol/L |
| Bicarbonate | 25 | mmol/L |
| Glucose | 2.27% | |
| Heparin | 2500 | IU/L |
| pH | 7.4 | |

### EXAMPLE 5

### PHYSIONEAL® with Non-Fractionated Heparin Added to Electrolyte

To determine the stability of non-fractionated Heparin in the electrolyte compartment of a commercially available bicarbonate/lactate buffered peritoneal dialysis solution (PHYSIONEAL® 40, 2000 ml), PHYSIONEAL(D was prepared and non-fractionated Heparin was added to result in a concentration of 2500 IU/L per bag. Immediately after addition of the non-fractionated Heparin, solutions with Heparin and solutions without Heparin were steam sterilized under standard conditions. For comparison, the same amount of non-fractionated Heparin was added through the medication port to the sterilized solution not yet containing Heparin after the sterilization process.

TABLE 6
uz,6/27 Composition of Experimental Solution Example 5

| Component | Concentration | |
|---|---|---|
| Sodium | 132 | mmol/L |
| Calcium | 1.25 | mmol/L |
| Magnesium | 0.25 | mmol/L |
| Chloride | 95 | mmol/L |
| Lactate | 15 | mmol/L |
| Bicarbonate | 25 | mmol/L |
| Glucose | 2.27% | |
| Heparin | 2500 | IU/L |
| pH | 7.4 | |

### Blood Clotting Test

The stability of Heparin in solutions with Heparin added before and after sterilization process was evaluated by measuring the activity of Heparin by the use of blood clotting test as described in the European Pharmacopeia 5.02 (Method 2.7.5).

### Results

The results for each of the experimental combinations as described above are shown in Tables 7-11.

TABLE 7
Results of Blood Clotting Test DIANEAL ® and Low Molecular
Weight Heparin

| Solution (DIANEAL ®) with LMWH | | | | |
|---|---|---|---|---|
| | Nominal Conc. | Activity IU/mL | | |
| Addition | IU/mL | mv* | N* | s* |
| before sterilization | 1.00 | 0.94 | 6 | 0.021 |
| after sterilization | 1.00 | 0.95 | 6 | 0.017 |
| before sterilization | 2.00 | 1.88 | 6 | 0.024 |
| after sterilization | 2.00 | 1.85 | 6 | 0.016 |
| before sterilization | 5.00 | 4.74 | 6 | 0.077 |
| after sterilization | 5.00 | 4.78 | 6 | 0.102 |

*mv: mean value; n: number of samples; s: standard deviation

TABLE 8
Results of Blood Clotting Test EXTRANEAL ® and Non-
Fractionated Heparin

| Solution (EXTRANEAL ®) with | | | | |
|---|---|---|---|---|
| Heparin | | | | |
| | Nominal Conc. | Activity IU/mL | | |
| Addition | IU/mL | mv* | n* | s* |
| before sterilization | 2.50 | 2.36 | 6 | 0.026 |
| after sterilization | 2.50 | 2.51 | 6 | 0.033 |

TABLE 9
Results of Blood Clotting Test EXTRANEAL ® and Low Molecular
Weight Heparin

| Solution (EXTRANEAL ®) with | | | | |
|---|---|---|---|---|
| Heparin | | | | |
| | Nominal Conc. | Activity IU/mL | | |
| Addition | IU/mL | mv* | n* | s* |
| before sterilization | 2.50 | 2.40 | 6 | 0.021 |
| after sterilization | 2.50 | 2.45 | 6 | 0.051 |

TABLE 10
Results of Blood Clotting Test PHYSIONEAL ® (buffer) and Non-
Fractionated Heparin

| Solution (PHYSIONEAL ®) with | | | | |
|---|---|---|---|---|
| Heparin | | | | |
| In buffer compartment | | | | |
| | Nominal Conc. | Activity IU/mL | | |
| Addition | IU/mL | mv* | n* | s* |
| before sterilization | 2.50 | 2.34 | 6 | 0.042 |
| after sterilization | 2.50 | 2.40 | 6 | 0.071 |

TABLE 11
Results of Blood Clotting Test (PHYSIONEAL ® (electrolyte) and
Non-Fractionated Heparin (Example A)

| Solution (PHYSIONEAL ®) with | | | | |
|---|---|---|---|---|
| Heparin | | | | |
| In electrolyte compartment | | | | |
| | Nominal Conc. | Activity IU/mL | | |
| Addition | IU/mL | mv* | n* | s* |
| before sterilization | 2.50 | 2.28 | 6 | 0.035 |
| after sterilization | 2.50 | 2.38 | 6 | 0.038 |

### Conclusion

Table 12 shows a summary of the ratio of Heparin added before sterilization to Heparin added after sterilization. The blood clotting tests show a variance of activity of 102% to 94% for the solutions containing Heparin before sterilization in comparison to the solutions where Heparin was added after sterilization. This variance indicates the stability of Heparins during the sterilization process. All described solution/Heparin combinations are stable under autoclaving and steam sterilization conditions as shown in the experimental examples previously described.

Moreover, in case of PHYSIONEAL®, it does not matter to which compartment the Heparin is added. Either in the acidic electrolyte compartment (pH˜4.2) or in the alkalic buffer compartment (pH˜7.5) the results are comparable.

TABLE 12
Comparison of Heparin Stability

| | | | Actual, | Actual, | |
|---|---|---|---|---|---|
| | | | added after | added before | before/ |
| | | Nominal | Steril. | Steril. | after |
| Solution | Heparin | (IU/ml) | (IU/ml) | (IU/ml) | Ratio (%) |
| Dianeal | LMWH | 1.00 | 0.95 | 0.94 | 99 |
| Dianeal | LMWH | 2.00 | 1.85 | 1.88 | 102 |
| Dianeal | LMWH | 5.00 | 4.78 | 4.74 | 99 |
| Extraneal | non frac | 2.50 | 2.51 | 2.36 | 94 |
| Extraneal | LMWH | 2.50 | 2.45 | 2.40 | 98 |
| Physioneal | non frac | 2.50 | 2.40 | 2.34 | 98 |
| buffer | | | | | |
| Physioneal | non frac | 2.50 | 2.38 | 2.28 | 96 |
| electrolyte | | | | | |

It should be understood that various changes and modifications to the presently preferred embodiments described herein will be apparent to those skilled in the art. Such changes and modifications can be made without departing from the spirit and scope of the present subject matter and without diminishing its intended advantages. It is therefore intended that such changes and modifications be covered by the appended claims.

The invention is claimed as follows:

1. A dialysis solution comprising a dialysis component and Heparin that are combined to form the dialysis solution, wherein the dialysis solution is sterilized after the dialysis component and the Heparin are combined.

2. The solution of claim 1, wherein the sterilization is done by a technique selected from the group consisting of autoclave, steam and combinations thereof.

3. The solution of claim 1, wherein the Heparin is selected from the group consisting of non-fractionated Heparin, low molecular weight Heparin, recombinant low molecular weight Heparin and combinations thereof.

4. The solution of claim 1, wherein the dialysis component is selected from the group consisting of osmotic agents, buffers, electrolytes and combinations thereof.

5. The solution of claim 4, wherein the osmotic agent is selected from the group consisting of glucose, glucose polymers, modified starch, hydroxyethyl starch, polyols, amino acids, peptides, glycerol and combinations thereof.

6. The solution of claim 4, wherein the buffer is selected from the group consisting of bicarbonate, lactate, pyruvate, acetate, citrate, an intermediate of the KREBS cycle and combinations thereof.

7. The solution of claim 1 comprising at least two dialysis components and wherein the Heparin is added to at least one of the dialysis components and sterilized with said dialysis component.

8. The solution of claim 7, wherein the two dialysis components are stored and sterilized separately.

9. The solution of claim 1, wherein the Heparin comprises a concentration in the dialysis solution from about 1000 IU/L to about 5000 Ru/L.

10. The solution of claim 1 comprising an acid selected from the group consisting of lactic acid/lactate, pyruvic acid/pyruvate, acetic acid/acetate, citric acid/citrate, an intermediate of the KREBS cycle, hydrochloric acid and combinations thereof.

11. A method of manufacturing a sterilized dialysis solution, the method comprising:
providing a dialysis component;
providing Heparin, wherein the Heparin is mixed with the dialysis component; and
sterilizing the mixture of the dialysis component and the Heparin.

12. The method of claim 1 1, wherein the sterilization is done by a technique selected from the group consisting of autoclave, steam and combinations thereof.

13. The method of claim 11, wherein the Heparin is selected from the group consisting of non-fractionated Heparin, low molecular weight Heparin, recombinant low molecular weight Heparin and combinations thereof.

14. The method of claim 11, wherein the dialysis component is selected from the group consisting of osmotic agents, buffers, electrolytes and combinations thereof.

15. The method of claim 14, wherein the osmotic agent is selected from the group consisting of glucose, glucose polymers, modified starch, hydroxyethyl starch, polyols, amino acids, peptides, glycerol and combinations thereof.

16. The method of claim 14, wherein buffer is selected from the group consisting of bicarbonate, lactic acid/lactate, pyruvic acid/pyruvate, acetic acid/acetate, citric acid/citrate, an intermediate of the KREBS cycle and combinations thereof.

17. The method of claim 11, wherein the dialysis solution comprises at least two dialysis components and wherein the Heparin is added to at least one of the dialysis components and sterilized with said dialysis component.

18. The method of claim 17, wherein the two dialysis components are stored and sterilized separately.

19. The method of claim 11, wherein the sterilized dialysis solution comprises an acid selected from the group consisting of lactic acid/lactate, pyruvic acid/pyruvate, acetic acid/acetate, citric acid/citrate, an intermediate of the KREBS cycle, hydrochloric acid and combinations thereof.

20. A method of manufacturing a sterilized solution, the method comprising:
providing at least one dialysis component;
adding a glycosaminoglycan to the dialysis component; and
sterilizing the dialysis component containing the glycosaminoglycan.

21. The method of claim 20 comprising at least two dialysis components, wherein the glycosaminoglycan is added to at least one of the dialysis components and sterilized with said dialysis component.

22. The method of claim 21, wherein the two dialysis components are combined to form a ready-to-use dialysis solution.

23. The method of claim 20, wherein the sterilization is done by a technique selected from the group consisting of autoclave, steam and combinations thereof.

24. The method of claim 20, wherein the glycosaminoglycan is selected from the group consisting of non-fractionated Heparin, low molecular weight Heparin, recombinant low molecular weight Heparin and combinations thereof.

25. The method of claim 20, wherein the dialysis component is selected from the group consisting of buffers, osmotic agents, electrolytes and combinations thereof.

26. The method of claim 25, wherein the osmotic agent is selected from the group consisting of glucose, glucose polymers, modified starch, hydroxyethyl starch, polyols, amino acids, peptides, glycerol and combinations thereof.

27. The method of claim 25, wherein the solution comprises an acid selected from the group consisting of lactic acid/lactate, pyruvic acid/pyruvate, acetic acid/acetate, citric acid/citrate, hydrochloric acid, an intermediate of the KREBS cycle and combinations thereof.

28. A method of providing dialysis to a patient in need of same, the method comprising:
providing an osmotic agent, a buffer and an electrolyte;
mixing Heparin with at least one of the osmotic agent, the buffer and the electrolyte to form a dialysis mixture;
sterilizing the dialysis mixture; and
providing a dialysis solution including the dialysis mixture to the patient.

29. The method of claim 28, wherein the sterilization is done by a technique selected from the group consisting of autoclave, steam and combinations thereof.

30. The method of claim 28, wherein the Heparin is selected from the group consisting of non-fractionated Heparin, low molecular weight Heparin, recombinant low molecular weight Heparin and combinations thereof.

31. The method of claim 28, wherein the osmotic agent is selected from the group consisting of glucose, glucose polymers, modified starch, hydroxyethyl starch, polyols, amino acids, peptides, glycerol and combinations thereof.

32. The method of claim 28, wherein the buffer is selected from the group consisting of bicarbonate, lactic acid/lactate, pyruvic acid/pyruvate, acetic acid/acetate, citric acid/citrate, an intermediate of the KREBS cycle and combinations thereof.

33. The method of claim 28, wherein the dialysis solution comprises an acid selected from the group consisting of lactic acid/lactate, pyruvic acid/pyruvate, acetic acid/acetate, citric acid/citrate, an intermediate of the KREBS cycle, hydrochloric acid and combinations thereof.

34. The method of claim 28, wherein the Heparin is mixed with all of the osmotic agent, buffer and electrolyte before sterilization.

35. A dialysis solution comprising a dialysis component and glycosaminoglycan that are combined to form the dialysis solution, wherein the dialysis solution is sterilized after the dialysis component and the glycosaminoglycan are combined.

36. The solution of claim 35, wherein the sterilization is done by a technique selected from the group consisting of autoclave, steam and combinations thereof.

37. The solution of claim 35, wherein the dialysis component is selected from the group consisting of buffers, osmotic agents, electrolytes and combinations thereof.

38. The solution of claim 37, wherein the buffer is selected from the group consisting of bicarbonate, lactate, pyruvate, acetate, citrate, an intermediate of the KREBS cycle and combinations thereof.

39. The solution of claim 35 comprising at least two dialysis components and wherein the glycosaminoglycan is added to at least one of the dialysis components and sterilized with said dialysis component.

40. The solution of claim 35, wherein the dialysis components are stored and sterilized separately.

41. The solution of claim 35, wherein the sterilized dialysis solution has a pH ranging from 4.5-8.

42. The solution of claim 35, wherein the glycosaminoglycan comprises a concentration in the dialysis solution from about 1000 IU/L to about 5000 IU/L.

43. The solution of claim 35 comprising an acid selected from the group consisting of lactic acid/lactate, pyruvic acid/pyruvate, acetic acid/acetate, citric acid/citrate, an intermediate of the KREBS cycle, hydrochloric acid and combinations thereof.
